(19) United States
(12) Patent Application Publication
(10) Pub. No.: US 2026/0047813 A1
(43) Pub. Date: Feb. 19, 2026

(54) SAFETY APPARATUS FOR ENABLING MOTION CONTROL COMMANDS

(71) Applicant: Siemens Healthineers AG, Forchheim (DE)

(72) Inventors: Verena SCHMIDT, Erbendorf (DE); Rolf SCHMIDT, Immenreuth (DE); Stefan DOLESCHAL, Grafenwoehr (DE)

(73) Assignee: Siemens Healthineers AG, Forchheim (DE)

(21) Appl. No.: 19/298,324

(22) Filed: Aug. 13, 2025

(30) Foreign Application Priority Data
Aug. 14, 2024 (DE) 10 2024 207 754.2

(51) Int. Cl. A61B 6/46; A61B 6/00

(52) U.S. Cl. CPC A61B 6/467; A61B 6/4476; A61B 6/547

(57) ABSTRACT

One or more example embodiments relates to a safety apparatus for enabling motion control commands for controlling a medical apparatus. The safety apparatus comprises a controller configured to control a movement of at least a component of the medical apparatus by an operator, wherein the controller includes a dead man's device configured to output dead man's information as a function of a state of the dead man's device; an operating sensor configured to output operating information as a function of an operation of the controller by the operator; and an evaluator configured to verify an enablement of a motion control command based on the dead man's information and the operating information.

### CROSS-REFERENCE TO RELATED APPLICATION(S)

The present application claims priority under 35 U.S.C. § 119 to German Patent Application No. 10 2024 207 754.2, filed Aug. 14, 2024, the entire contents of which are incorporated herein by reference.

### FIELD

One or more example embodiments relates to a safety apparatus for enabling motion control commands for controlling a medical apparatus.

### RELATED ART

In the field of medical technology, in particular medical imaging apparatus, procedures are increasingly being automated. This is the case not only for fixed apparatuses, such as for example magnetic resonance apparatuses, but also, to a greater extent, mobile X-ray devices, i.e., X-ray devices displaceable in space such as for example C-arm X-ray devices. Such apparatuses therefore increasingly comprise multiple moving components, in particular with suitable drive platforms which allow automatable movement of the components in space.

In order, for example, to capture an X-ray image with a medical imaging apparatus, various components of the X-ray apparatus, for example the X-ray detector and X-ray emitter, have to be oriented. For this purpose, the X-ray detector and/or X-ray emitter may, for example, be arranged adjustably on a ceiling bracket and oriented in various degrees of freedom (conventionally up to three translational and three rotational axes), such that the X-ray emitted by the X-ray emitter passes through the patient at the correct position and correct angle for the region of the body to be examined and impinges fully on the X-ray detector. Positioning of the ceiling bracket may be performed manually by an operator, or may, in particular, be motor-driven. However, in particular when movement is motor-driven there is a risk, in principle, of the patient, the operator and/or other people present in the space, or components of the medical apparatus, for example patient couches, radiation protection apparatuses, assistance apparatuses, ECG or ultrasound devices, infusion stands and the like being injured or damaged by collision with the ceiling bracket or the X-ray emitter.

The operator of the medical apparatus thus has to enable and visually monitor movement in order to ensure safe movement of the component, for example of the X-ray emitter, in the movement space. To this end, not only are movement actuation operator interfaces known, and/or control units with which component movement can be defined and/or set, but also in particular enabling switches, also designated “dead man's switches” or “dead man's grips”. To enable movement it is known to hold such an enabling switch constantly in an enabling position. Collision between components can be reliably prevented by letting go of said switch.

However, given the large number of components and/or tasks an operator has to perform while operating a medical apparatus, the operator cannot always be sure of keeping all, especially moving, components of the medical apparatus in view. For example, the operator has continuously to ensure that the movement path (trajectory) of a ceiling bracket, for example, is free, so as to be able to move it unimpeded from the starting position to the desired target position. To this end, the operator has to know and/or be able to estimate the trajectories of the ceiling bracket, so as to be able to assess where people or objects can be situated in the space without the ceiling bracket colliding with them during automated and/or motor-driven movement. In particular, the operator has themselves to be positioned such that they are not in a movement path (trajectory) of a component during movement thereof. If this occurs, however, and the operator lets go of the enabling switch, movement of the component stops immediately. The operator is thus in a position to identify an impending collision for example between a ceiling bracket and a person, in particular themselves, and avert it by letting go of the enabling button. It is also possible for an operator to suffer cramp on an enabling switch due to extended operation thereof and/or in the event of a critical situation, so meaning that they can no longer avert an impending collision by letting go of the enabling switch. Due to the increasing automation of procedures, it may additionally happen that the exact movement path of a component is unknown to the operator. This leads to a complex environment around the medical apparatus with dynamic obstacles for which known enabling switches constitute an inadequate solution.

### SUMMARY

One or more example embodiments provides an operator of a medical apparatus with a reliable enabling means for controlling the movement of components in an environment with dynamic obstacles and hazards.

### BRIEF DESCRIPTION OF THE DRAWINGS

Advantages, features and details are revealed by the exemplary embodiments described below with reference to the drawings. Mutually corresponding parts are provided with the same reference numerals in all the figures. No repeated description of mutually corresponding parts in the respective exemplary embodiments is given. Exemplary embodiments may differ substantially in the arrangement of the units.

In the drawings:

FIG. 1 shows a schematic representation of a safety apparatus for enabling motion control commands for controlling a medical apparatus according to one or more example embodiments,

FIG. 2 shows a schematic representation of an embodiment of the safety apparatus for enabling motion control commands for controlling a medical apparatus according to one or more example embodiments,

FIG. 3 shows a medical apparatus comprising a safety apparatus for enabling motion control commands for controlling the medical apparatus according to one or more example embodiments,

FIG. 4 shows an embodiment of a medical apparatus comprising a safety apparatus,

FIG. 5a shows a schematic representation of a safety apparatus with a capacitive dead man's device in the idle state according to one or more example embodiments,

FIG. 5b shows a schematic representation of a safety apparatus with a capacitive dead man's device in the enabling state according to one or more example embodiments,

FIG. 5c shows a schematic representation of a safety apparatus with a capacitive dead man's device in the blocking state according to one or more example embodiments,

FIG. 6 shows a schematic representation of a safety apparatus with a mechanical dead man's device in the enabling state according to one or more example embodiments, and

FIG. 7 shows a representation of the correlation between the dead man's information and the operating information of the safety apparatus as a characteristic curve according to one or more example embodiments.

### DETAILED DESCRIPTION

A safety apparatus is proposed for enabling motion control commands for controlling a medical apparatus. The safety apparatus comprises a control unit for motion control of the medical apparatus by an operator. The control unit comprises a dead man's device. The dead man's device is configured to output dead man's information as a function of a state of the dead man's device. The safety apparatus comprises an operating sensor unit. The operating sensor unit is configured to output operating information as a function of operation of the control unit by the operator. The safety apparatus comprises an evaluation unit. The evaluation unit is configured to verify enabling of a motion control command on the basis of the dead man's information and the operating information.

In particular, the control unit may be configured to control movement of a component of the medical apparatus. The dead man's device may in particular be configured to output dead man's information as a function of an instantaneous state of the dead man's device. The evaluation unit is configured in particular to verify enabling of a motion control command for controlling movement, in particular of the component, of the medical apparatus on the basis of the dead man's information and the operating information.

The motion control command makes it possible, in particular, to control movement of a component of a medical apparatus. The control unit of the medical apparatus is preferably configured to acquire the motion control command and to control movement of a component of the medical apparatus in accordance with the motion control command. The control command in particular comprises an instruction or a signal which is sent to a component of an apparatus, a system or a unit to perform specific actions and/or processes. These control commands may, for example, relate to starting up, stopping, accelerating or slowing down a component (and are therefore designated motion control commands).

The medical apparatus may in particular comprise a medical imaging apparatus and/or a medical treatment apparatus. Medical imaging systems may comprise one or more imaging modalities such as, for example, computed tomography devices, magnetic resonance devices, X-ray devices, ultrasound devices and the like. In particular, the medical system may be a medical treatment apparatus. In particular, the medical system may be or comprise an irradiation device.

In particular, the medical apparatus may comprise a mobile medical device which is displaceable in space using suitable (drivable) movement means and in the process changes its position in space. For example, a mobile medical device may be an imaging C-arm X-ray device with (drivable) wheels, which is displaceable from one spatial position into another spatial position.

The medical apparatus may in particular comprise multiple components. In particular, multiple components of the medical apparatus may be movable. In particular, stationary medical devices may have components which are movable between defined spatial positions. For example, one component may be an adjustable, displaceable patient couch.

The operator of the medical apparatus may in particular be a medically trained person, a person trained in operating the medical apparatus, medical personnel and/or a physician.

Movement of a medical apparatus, in particular a component of a medical apparatus, may preferably be controlled using an interface, in particular a human-machine interface of the control unit of the medical apparatus and/or a movement actuation operator interface. In particular, the interface of the control unit may be configured to plan a displacement path, to display a displacement path to the operator and/or to control movement of a medical apparatus, in particular of a component of a medical apparatus.

Displacement path planning by an operator may in particular comprise defining a start position and/or target position. Furthermore, displacement path planning by an operator may preferably comprise a trajectory, a route, a distance, a path, a travel way and/or a movement path. The displacement path to be displayed to the user and optionally selected and/or confirmed by the user is preferably determined autonomously by the medical apparatus, in particular the control unit. The control unit may likewise be denoted a computing unit and/or evaluation unit and/or comprise such a unit. The movement and/or displacement path of the medical apparatus, in particular of a component, may, however, also be calculated in a computing unit outside the medical apparatus, for example in a space detection system. The dead man's device of the safety apparatus may allow an operator to enable the calculated movement, in particular the motion control command.

In addition to the dead man's device, the control unit of the safety apparatus preferably also comprises a human-machine interface, in particular for the control and/or computing unit of the medical apparatus. The control unit of the safety apparatus may in particular be encompassed by the control and/or computing unit of the medical apparatus. The control unit of the safety apparatus may preferably be configured to receive a motion control command from a control and/or computing unit of the medical apparatus. The control unit of the safety apparatus may preferably comprise the evaluation unit. If the motion control command is enabled by verifying the dead man's information and the motion information, the control device of the safety apparatus may in particular be configured to output the motion control command to the relevant component of the medical apparatus and/or to a control and/or computing unit of the medical apparatus.

The dead man's device may in particular comprise a motionless detector, dead man, dead man's alarm, dead man's switch, dead man's pedal, dead man's button and/or dead man's detector. The dead man's device may preferably comprise an operating element. The operating element may preferably allow an operator to perform an input, in particular in the form of enabling a command. The operating element may, for example, comprise an operating head, button, switch, grip, or pedal. In particular, a dead man's device may be configured to verify whether an operator is present and/or ready for action. Furthermore, the dead man's device may be used by an operator to confirm and/or monitor movement of the medical apparatus constantly and/or over the duration of the movement of the medical apparatus. In other words, movement of the medical apparatus and/or a component may proceed providing the operator is actuating, for example pressing, the dead man's device.

The operating sensor unit may in particular comprise a pressure sensor, capacitive sensor, force sensor, Hall effect sensor and/or optical sensor. In particular, the operating sensor unit may comprise a plurality of sensors for acquiring operating information. In particular, the operating sensor unit may be configured to detect operation of the control device, in particular of an operating element of the control device, by the operator, in particular by ascertaining a force with which the operator is operating the control device and to output this as operating information. The operating sensor unit may in particular be encompassed by the dead man's device. In particular, the operating sensor unit may be arranged on an operating element of the dead man's device and/or be encompassed thereby. The operating sensor unit may in particular be configured to convert a non-electrical measured variable relating to operation of the control device by the operator into the operating information, in particular an electrical signal. In particular, an operating element of the control device may comprise the operating sensor unit. A capacitive sensor may, for example, be used to ascertain a change in capacitance due to the application of force (pressure) by the operator to an operating element of the control device.

The operating information may, in particular, comprise information that characterizes and/or describes operation of the control device. In particular, the operating information may comprise a measured variable for a force exerted by the operator, in particular a force (pressure) exerted on a surface. In particular, the operating information may comprise information relating to the direction of a force exerted by the operator. In other words, the operating information may in particular comprise information that characterizes the intensity and direction of a force applied during operation of the control device by the operator.

The evaluation unit is preferably configured to receive and evaluate the operating information and the dead man's information. The evaluation unit preferably comprises a computing unit that is configured to perform an enable verification method. In particular, the computing unit is configured to verify whether the operator and/or other people, such as for example a patient, is/are in a hazardous situation on the basis of the dead man's information and the operating information.

The safety apparatus may advantageously allow safe operation of the medical apparatus by the operator. In particular, the safety apparatus may ensure operator safety, in particular by preventing collisions between the operator and components of the medical apparatus. The safety apparatus is advantageously preferably designed so as to allow the operator to handle the safety apparatus in a manner similar to the known manner of handling an enabling switch. In addition, the safety apparatus may in particular allow simplified monitoring of a complex, dynamic operating situation.

In one preferred embodiment, the safety apparatus comprises a motion detection apparatus. The motion detection apparatus is configured to output motion information as a function of operator movement. The evaluation unit is configured additionally to verify enabling of a motion control command on the basis of the motion information.

The motion detection apparatus may in particular comprise a motion detector and/or a camera. In particular, the motion detection apparatus may be configured to detect operator movements in an environment, in particular a movement region, and output these as motion information. The motion detection apparatus may be configured to ascertain operator movement by emitting and/or receiving electromagnetic waves, ultrasound and/or infrared radiation. Furthermore, acceleration and strain rate sensors may be encompassed by the motion detection apparatus.

The motion information preferably comprises spatial positioning of the operator relative to a reference object or reference point. The reference object is preferably an immobile component of the medical apparatus. The motion information may in particular comprise two-dimensional and/or three-dimensional movement data, in particular relating to the operator.

The motion detection apparatus may additionally be configured to detect the movements of further people, such as for example a patient, and/or of the medical apparatus, in particular of the components thereof. In particular, the motion detection apparatus may be positioned in such a way within the medical apparatus and/or a space in which the medical apparatus is positioned that the motion detection apparatus may detect movements within the medical apparatus and/or the space, in particular in a movement region. The motion detection apparatus may, for example, be configured as a swivelable camera arranged on the ceiling in the middle of a space encompassing a medical apparatus.

Movement may in particular be understood to mean a change in the spatial positioning of a person and/or a component in particular on a movement trajectory and/or path. The motion detection apparatus may in particular be configured to ascertain movement of the operator and/or of the medical apparatus in a movement region. The movement region may, for example, be a region in which an operator may stay to operate the medical apparatus. The movement region may in particular be delimited by components arranged in a stationary manner, such that the motion detection apparatus may be configured and/or arranged in accordance with the extent of the movement region.

The evaluation unit is preferably additionally configured to receive and evaluate the motion information. In particular, the computing unit is configured to verify whether the operator and/or other people, such as for example a patient, is/are in a hazardous situation on the basis of the motion information.

In particular, the safety apparatus may ensure operator safety, in particular by preventing collisions between the operator when said operator is moving and components of the medical apparatus. The safety apparatus is advantageously in particular designed so as to allow operator movement to be ascertained in particular during handling of the safety apparatus. In addition, the safety apparatus may in particular allow simplified monitoring for the operator if a complex, dynamic operating situation requires constant operator movement.

In one embodiment of the safety apparatus, provision is made for the motion detection apparatus to be so configured as to acquire three-dimensional operator movement data and to output the acquired three-dimensional movement data as motion information.

Three-dimensional movement data may in particular comprise information about the movements of objects and/or people in the three-dimensional space. In particular, the three-dimensional movement data may vary dynamically and continuously over time. In particular, the three-dimensional movement data may be ascertainable by way of optical movement analysis by the motion detection apparatus. In this case, information may preferably be detected by the motion detection apparatus via markers on the operator, in particular on the operator's clothing, and the movement and/or position of the operator may be calculated from this information. However, marker-less detection of operator movement is also conceivable, in particular on the basis of analysis of an image series, in particular by way of a trained function.

Acquisition of three-dimensional operator movement data by the motion detection apparatus may allow the output of accurate motion information. The acquisition of three-dimensional movement data may advantageously allow extensive spatial detection of situations hazardous to the operator.

For example the detection of hazardous situations, which cannot be detected using in particular two-dimensional movement data, can be enabled.

In one preferred embodiment of the safety apparatus, the motion detection apparatus comprises a portable sensor unit. The portable sensor unit may preferably be worn by the operator during operation of the medical apparatus. The portable sensor unit may in particular allow accurate operator position determination in a space and/or the acquisition of three-dimensional movement data. The motion detection apparatus may in particular comprise a further detection sensor unit, for example a camera. In particular, the combination of various detection sensor units encompassed by the motion detection apparatus may allow an accurate description of the movement of an operator and/or the output of comprehensive motion information.

In one embodiment of the safety apparatus, provision is made for the evaluation unit to be so configured as to receive apparatus position data and to verify enabling of the motion control command on the basis of the apparatus position data. The apparatus position data in particular describes a movement of the medical apparatus.

In particular, the apparatus position data describes a movement of a component of a medical apparatus.

The apparatus position data may preferably be transmitted by a control and/or computing unit of the medical apparatus to the evaluation unit. In particular, the apparatus position data may be ascertained using one or more sensor units. To this end, the sensor unit may in particular be arranged on the components of the medical apparatus and/or encompassed thereby. A global sensor unit for detecting movement of the components of the medical apparatus is additionally conceivable, in particular arranged centrally within the medical apparatus. In particular, apparatus position data may comprise three-dimensional movement data of the medical apparatus, in particular of a component.

In particular, a motion detection apparatus may be configured to output apparatus position data. In particular, the motion detection apparatus may be configured to acquire the movement of the medical apparatus and/or of a component of the medical apparatus.

Verification by the evaluation unit of motion control command enabling may in particular comprise comparing operator motion information and/or operating information with apparatus position data. In particular, the apparatus position data may be used by the evaluation unit to verify whether a movement path of one component of the medical apparatus interferes with a movement path of a further component of the medical apparatus. In particular, the evaluation unit may be used to verify whether a movement path of the operator interferes with a movement path of the medical apparatus and/or of a component of the medical apparatus.

Verification of motion control command enabling on the basis of apparatus position data may allow the movement paths of the medical apparatus, in particular plurality of multiple components of the medical apparatus, to be taken into account.

In one embodiment of the safety apparatus, provision is made for the evaluation unit to be so configured as to detect the presence of a hazardous situation for the operator on the basis of the motion information and the apparatus position data. In the case of a hazardous situation, the evaluation unit is configured to output an emergency stop control command.

Detection of a hazardous situation for the operator may in particular comprise the evaluation unit comparing the motion information and apparatus position data. In particular, the comparison of motion information and apparatus position data may be used by the evaluation unit to verify whether an operator movement path interferes with a movement path of the medical apparatus and/or of a component of the medical apparatus. Such a movement path overshoot and/or an impending collision, for example between a component and the operator, may be designated a hazardous situation.

An emergency stop control command may in particular comprise a control command at any, preferably mobile, components of the medical apparatus. In particular, an emergency stop control command may be used to immediately stop all movements of components of the medical system and/or of the medical system. In other words, the medical apparatus may be configured to switch into a stationary and/or switched-off state in the presence of an emergency stop control command.

The detection of hazardous situations makes it advantageously possible to improve safety for the operator of the medical apparatus. By detecting hazardous situations, it may additionally be possible to prevent serious collisions.

In one embodiment of the safety apparatus, provision is made for the dead man's device to be so configured as to generate dead man's information as a function of at least three states of the dead man's device.

Dead man's devices comprise in particular two states, in particular switching states and/or positions. The states of the dead man's device may in particular allow an operator to operate a medical apparatus. As a result of the various states, it is possible for an operator for example not only to enable and/or initiate a movement, in particular a motion control command, but also to interrupt a movement, in particular a motion control command, and/or initiate an emergency stop control command.

In particular, a third state of the dead man's device, or a dead man's device with three states, may allow operation of the medical apparatus to be improved for the operator and operating safety to be increased for the operator.

In one embodiment of the safety apparatus, provision is made for the control unit to comprise an operating element.

One of the following states of the dead man's device is settable by way of the operator's operating intensity of the operating element:
an idle state,
an enabling state,
a blocking state.

The dead man's device exhibits the idle state if the operating intensity is below a first specified limit value. The dead man's device exhibits the enabling state if the operating intensity is between the first limit value and a second specified limit value. The dead man's device exhibits the blocking state if the operating intensity exceeds the second limit value. The control unit is configured to output an emergency stop control command when the dead man's device is in the blocking state.

In particular, the dead man's device of the control unit comprises an operating element. The operating element may in particular comprise a grip or a button. The operating intensity may in particular describe a force (and/or impetus) with which the operator acts on the operating element of the dead man's device. The dead man's device may preferably be configured to ascertain the operating intensity, in particular as a discrete value. The dead man's device may preferably be configured to verify whether the operating intensity exceeds and/or falls below predetermined limit values, and to switch to a corresponding state. The dead man's device may, for example, be configured to ascertain the operating intensity using a mechanical resistance, in particular a spring resistance, and/or an electrical resistance, in particular a capacitive resistance.

The idle state in particular constitutes a first state, an initial state, an off state and/or a standard state of the dead man's device. The idle state of the dead man's device prevails in particular when the dead man's device is not being operated by an operator, in particular no mechanical force is being exerted by the operator on the dead man's device, in particular on the operating element of the dead man's device. The dead man's device may for example be configured such that, if the operator is no longer holding the operating element of the dead man's device in their hand and/or not actuating it continuously, the dead man's device switches immediately into the idle state. The dead man's device may in particular be configured such that switching to the idle state has to happen after output of an emergency stop control command and/or after emergency stoppage of the medical apparatus.

The enabling state in particular constitutes a second state of the dead man's device. The enabling state is in particular the state with which the operator may enable movement of the medical apparatus, in particular a motion control command. The dead man's device may preferably be configured such that the enabling state of the dead man's device may be set by continuous or periodic actuation of the operating element of the dead man's apparatus.

The blocking state in particular constitutes a third state of the dead man's device. The blocking state of the dead man's device may in particular be a state which is not perceived (and/or selectable) by the operator. In other words, the blocking state may in particular be a state which cannot be selected by the operator in normal operation, and/or to which the operator may switch by atypical operation of the dead man's device, for example cramp. The blocking state of the dead man's device may in particular be a safety state in which no movement of the medical apparatus takes place. Such a safety state, in particular blocking state, for example allows operator cramp and/or an operator's incapacity to act to be identified. In other words, the dead man's device may in particular switch into the blocking state if a maximum operating intensity value is exceeded.

The blocking state of the dead man's device may however also be a state selectable by the operator and/or a switchable state. For example, a movement may be enabled in particular by an enabling state and stopped briefly by a third state without the movement, in particular of a component of a medical apparatus, having to be interrupted.

A safety apparatus configured in this way advantageously allows convenient, intuitive and simplified operation for the operator of the medical apparatus. Operator cramp and/or operator incapacity to act may in particular advantageously be acquired by the blocking state of the dead man's device and a countermeasure, in particular outputting of an emergency stop control command, may be automatically initiated.

In one embodiment of the safety apparatus, provision is made for the motion control command to be enabled by the evaluation unit only if the dead man's device is in the enabling state.

If the motion control command is enabled by the operator setting an enabling state, this in particular makes it simpler for the operator to handle the medical apparatus. In particular, an enabling state which has to be continuously confirmed by the operator ensures that the operator continues to monitor movement of the medical apparatus, in particular of a component.

In one embodiment of the safety apparatus, provision is made for the safety apparatus to comprise an output unit. The output unit is configured to output a warning signal to the operator in the presence of the blocking state.

In particular, the warning signal may comprise a light and/or audio signal. In particular, the warning signal may be used to output the blocking state to the operator and/or to elucidate stoppage of the device and/or a hazardous situation. In particular, the output unit may be configured to output an announcement signal at a specific time interval prior to the warning signal. In particular, inadvertent activation of the blocking state may in this case be signaled to the operator and corrected by the operator.

The warning signal may in particular comprise one or more sounds and/or sound sequences. The sounds may preferably be output at different volumes and/or frequencies by a loudspeaker of the output unit. The warning signal may additionally comprise an optical signal, in particular a light signal. In particular, the output unit may comprise a warning light, which may output a warning signal in the form of a colored light signal.

In particular, the output unit may comprise a vibration generation unit. The warning signal may in particular comprise vibration, preferably of an operating element of the control device, in particular of the dead man's device.

In particular, output of a warning signal by the output unit allows the transmission of information, in particular a warning, to the operator in a hazardous situation, for example were the operator to get cramp.

In one embodiment of the safety apparatus, provision is made for the operating element of the dead man's device to comprise the operating sensor unit. The operating sensor unit is configured to acquire the operator's operating intensity of the operating element. In particular, the sensor unit may comprise a pressure sensor.

In particular, the operating sensor unit may be configured to transform a (mechanical) pressure and/or force applied by the operator to a surface of an operating element into an electrical output variable. In particular, the operating sensor unit may be configured as a pressure sensor, in particular as a piezoresistive or piezoelectric pressure sensor. Furthermore, the operating sensor unit, in particular the pressure sensor, may be configured to determine changes to operator's operating intensity of the dead man's device, in particular by measuring pressure differences and/or pressure variations.

The operating information may preferably comprise the operating intensity of the dead man's device. The operating intensity of the dead man's device may preferably be output by the sensor unit as a discrete value, in particular in pascals. In particular, the operating sensor unit may be configured to initiate a switch in the state of the dead man's device by determining operating intensity and checking operating intensity against predetermined threshold values.

Determination of the operating intensity of the dead man's device by the operating sensor unit may in particular allow the dead man's device to be adapted to the operator. For example, the operating intensity limit values may be set to correspond to the operator. Additional acquisition of the operating intensity by a sensor unit, in particular redundant in relation to acquisition of the operating intensity of the dead man's device, may increase operating safety.

In one embodiment of the safety apparatus, provision is made for the dead man's device to comprise a capacitive switch. The capacitive switch is configured to acquire an operating capacitance of the dead man's device.

The dead man's device may alternatively or additionally comprise a mechanical switch.

The capacitive switch may preferably comprise two opposingly arranged elements, in particular electrically conductive plates, and/or an arrangement of capacitors. The spacing of the arranged elements of the capacitive switch preferably defines operating capacitance. Furthermore, the capacitive switch may also be designated a capacitive sensor. The spacing of the elements, in particular, and thus the operating capacitance, in particular an electrical capacitance, and/or a variation of the latter may be determined as a function of operation of the dead man's device by the operator.

A capacitive switch may in particular allow states of the dead man's device to be defined by way of capacitive resistance. In particular, a capacitive switch may allow operation of the dead man's device which is imperceptible to the operator. In particular, this may improve and/or simplify operation of the dead man's device.

In one embodiment of the safety apparatus, provision is made for the evaluation unit to be so configured as to receive motion information, operating information and dead man's information and apparatus position data on verification of enabling of the motion control command. Furthermore, the evaluation unit is configured to verify a hazardous situation for the operator on the basis of the motion information, the operating information and the apparatus position data on verification of enabling of the motion control command. The motion control command is enabled by the evaluation unit if the situation for the operator is not hazardous and the dead man's device is in the enabling state.

In particular, the safety apparatus may advantageously allow safe operation of the medical apparatus by the operator while taking account of the movements of the operator and the medical apparatus. In particular, this may allow simplified operation in complex and/or dynamic operating situations.

In one embodiment of the safety apparatus, provision is made for the safety apparatus to comprise an output unit. The output unit is configured to inform the operator of enabling of the motion control command by way of an enable signal.

The enable signal may in particular comprise a light and/or audio signal. In particular, the enable signal may be used to output enabling of a motion control command to the operator and/or to elucidate the onset of an enabled movement. The enable signal may in particular comprise one or more sounds and/or sound sequences. The sounds may preferably be output at different volumes and/or frequencies by a loudspeaker element of the output unit. In particular, the output unit may comprise a light source, which may output an enable signal in the form of a colored light signal. The output unit may, for example, comprise two light sources, which are configured in each case to output an enable signal and a warning signal. The output unit may, for example, be configured as a traffic light device.

In particular, output of an enable signal, in particular a light signal and/or audio signal, by the output unit allows the transmission of information about a movement to the operator. Detection of the time of enabling by the enable signal may allow the operator to operate the medical apparatus in an improved manner.

Furthermore, a medical apparatus is proposed which comprises a safety apparatus according to one of the above embodiments.

In particular, the safety apparatus may be encompassed by a component of the medical apparatus. In particular, the safety apparatus may comprise two or more safety apparatuses.

The medical apparatus shares the advantages of one of the above-described embodiments. In particular, the medical apparatus comprising a safety apparatus according to one of the embodiments may increase safety for the operator.

In one embodiment of the medical apparatus, provision is made for the control unit to be configured to stop movement of the medical apparatus on output of an emergency stop control command by the safety apparatus.

In particular, the control and/or computing unit of the medical apparatus may comprise the control unit of the safety apparatus. In particular, the control unit may be configured to switch off the medical device on output of an emergency stop control command by the safety apparatus and/or to switch it into an operating state in which the medical apparatus does not move.

In particular, immediate stoppage of all movements of the medical apparatus may mean a collision is avoided in a hazardous situation. In particular, this may lead to increased safety of the medical apparatus.

In one embodiment of the medical apparatus, the medical apparatus comprises a sensor unit configured to ascertain apparatus position data.

In particular, a sensor unit of the medical apparatus may allow accurate apparatus position data to be ascertained at any point in time, for example for a component of the medical apparatus. In particular, this may additionally and advantageously allow more precise, more efficient and safer operation of the medical apparatus.

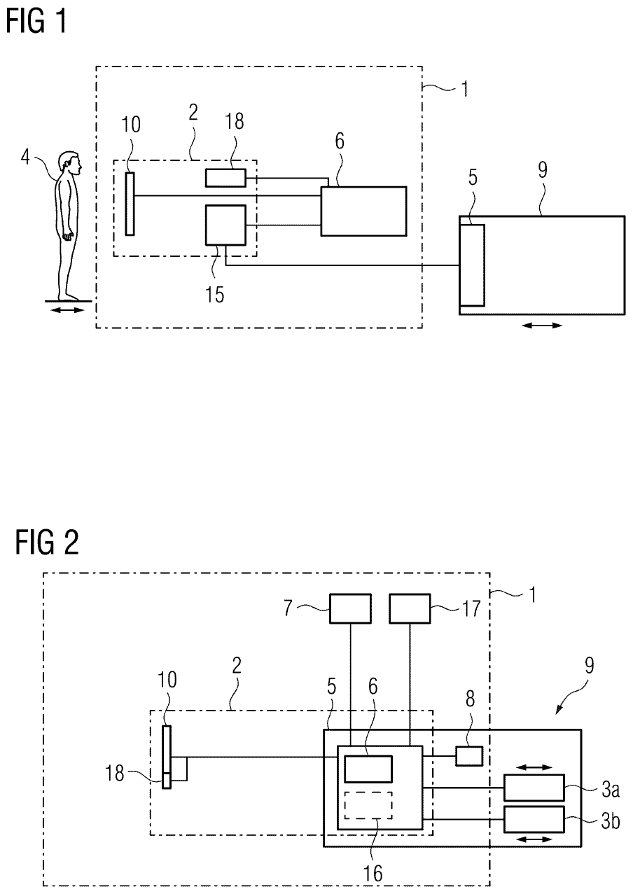

FIG. 1 is a schematic representation of a safety apparatus 1 for enabling motion control commands for controlling a medical apparatus 9. The safety apparatus 1 comprises a control unit (also referred to as a controller) 2 comprising a dead man's unit 10 and an interface 15. The interface 15 may in particular, as illustrated, be the interface with a control unit (also referred to as a controller) 5 of the medical apparatus 9. The control unit 2 of the safety apparatus 2 may allow an operator 4 to operate the medical apparatus 9. The interface 15 of the medical apparatus 9 may comprise a user interface which may allow the operator 4 to input a motion control command. The safety apparatus 1 additionally comprises an operating sensor unit (also referred to as an operating sensor) 18, and an evaluation unit 6 (also referred to as an evaluator). The control unit 2 comprises the operating sensor unit 18. The operating sensor unit 18 may be configured to acquire the operating information (force intensity and force direction with which the operator operates the dead man's device 10) of the operator 4. The dead man's device 10 may transmit dead man's information to the evaluation unit 6 as a function of operation by the operator 4. The evaluation unit 6 may be configured to receive operating information from the operating sensor unit 18 and dead man's information from the dead man's device 10 and to verify enabling of the motion control command to the medical apparatus 9. If the result of the verification is enabling of the motion control command, the motion control command may be transmitted to the control unit 5 of the medical apparatus 9 by way of the interface 15.

FIG. 2 is a schematic representation of a further embodiment of the safety apparatus 1 for enabling motion control commands for controlling a medical apparatus 9. Unlike in FIG. 1, the control unit 2 of the safety apparatus 1 comprises the control unit 5 of the medical apparatus 9. In addition, the safety apparatus 1 comprises a motion detection apparatus (also referred to as a motion detector) 7. The motion detection apparatus 7 may be configured to acquire the operator's movements. The motion detection apparatus 7 may transmit the detected movements of the operator 4 as motion information and/or the detected movements of the medical apparatus 9 to the evaluation unit 6 as apparatus position data. In addition, the evaluation unit 6 and optionally a computing unit 16 may be encompassed by the control unit 5 and/or the control unit 2. The evaluation unit 6 may be configured to receive motion information and/or apparatus position data from the motion detection apparatus 7 and dead man's information from the dead man's device 10 and to verify enabling of the motion control command to the medical apparatus 9. The medical apparatus 9 comprises two movable components 3a and 3b. The safety apparatus 1 additionally comprises an output unit 8. The output unit 8 may be a component of the medical apparatus 9 and/or be encompassed thereby. The safety apparatus 1 (or unlike in the illustration: the medical apparatus 9) may additionally comprise a sensor unit 17. The sensor unit 17 may be configured to transmit apparatus position data for components 3a, b of the medical apparatus 9 to the evaluation unit 6 or the control unit 5. The sensor unit 17 may be configured to ascertain the movements of components 3a, b of the medical apparatus 9. The computing unit 16 may be configured to ascertain motion control commands and provide them to the control unit 5. For example, on the basis of the imaging protocol in the event of the medical apparatus 9 being an imaging apparatus, the computing unit 16 may calculate the movements of components 3a, b required for an imaging examination and provide them as a motion control command to the evaluation unit 6 of the control unit 5. The evaluation unit 6 may verify enabling of the motion control command for example by using the apparatus position data transmitted by the sensor unit 17 and/or the motion information provided by the motion detection apparatus 7 and the dead man's information provided by the dead man's device 10 and the operating information provided by the operating sensor unit 18. The operating sensor unit 18 is encompassed by the dead man's device 10.

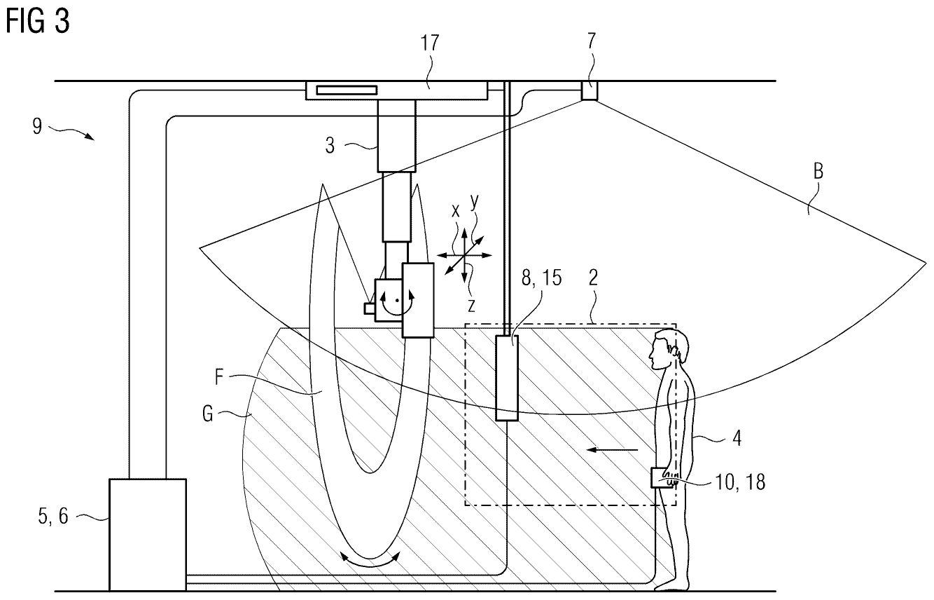

FIG. 3 shows a medical apparatus 9 comprising a safety apparatus for enabling motion control commands for controlling the medical apparatus 9. The evaluation unit 6 of the safety apparatus is encompassed by the control unit 5 of the medical apparatus 9. The dead man's unit 10 comprising the operating sensor unit 18 of the safety apparatus is encompassed by the control unit 2 of the safety apparatus. The control unit 2 allows the operator 4 to input a motion control command, in particular by way of an interface 15. The dead man's device 10 may be configured such that an operator can hold the dead man's device 10 in one hand. In addition, the dead man's device 10 may comprise an operating element (not shown) that allows the operator 4 to set a state for the dead man's device 10. The operating element that is not shown may comprise the operating sensor unit 18. The control unit 2 of the safety apparatus may transmit a motion control command to the control unit 5 of the medical apparatus 9; however, said command can only be executed once enabling has been verified using the evaluation unit 6 with enabling as the result. The interface 15 may be encompassed by an output unit 8. The component 3 of the medical apparatus 9 comprises a sensor unit 17. The sensor unit 17 is preferably configured to track the movements of component 3, in particular within a movement region F, and to transmit apparatus position data to the evaluation unit 6. The motion detection apparatus 7 is configured, in particular in a region B, to track movements of the operator 4, in particular in a movement region G, and to transmit motion information to the evaluation unit 6. The evaluation unit 6 may be configured to determine the movement region G of the operator 4 and/or the movement region F of the component 3 on the basis of the motion information and/or apparatus position data. If movement regions G and F overlap, a hazardous situation for the operator 4 may arise, such that in this case the evaluation unit 6 would not enable the motion control command. In addition, the evaluation unit may output an emergency stop command in a hazardous situation. The medical apparatus 9 may comprise an output unit 8, which is configured to inform the operator 4 of a hazardous situation and/or of the execution of an emergency stop control command.

For example, the medical device 1 may be an X-ray and/or radiation therapy apparatus comprising a radiation therapy head and/or an X-ray head as the component 3 orientable in space. The radiation therapy apparatus may, for example, comprise a monitor as output unit 8 which may be arranged adjustably on the ceiling of a room comprising the radiation therapy apparatus. A risk to an operator 4 of the radiation therapy apparatus may arise merely if their distance from the radiation therapy head falls below a given distance. The safety of the operator 4 may be increased, for example, by a safe distance from the radiation therapy head being ensured through the movement of the operator being acquired by the motion detection apparatus 7 and/or the evaluation unit 6.

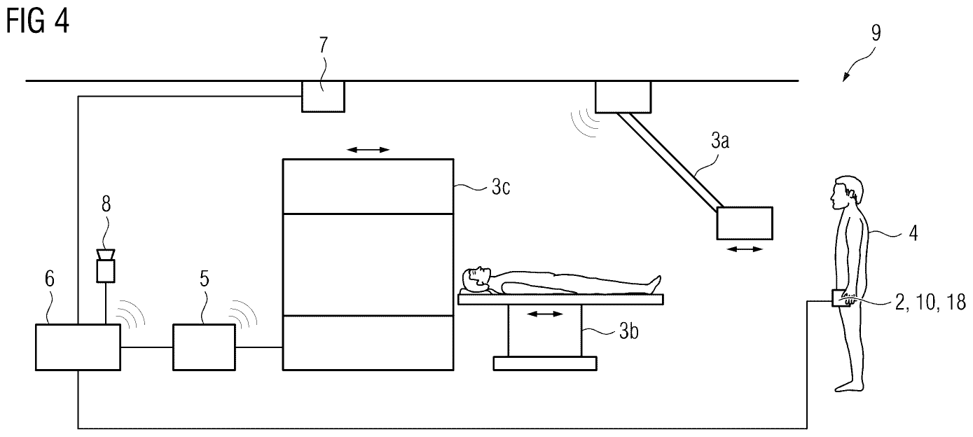

FIG. 4 shows a magnetic resonance apparatus as the medical apparatus 9 comprising a safety apparatus for enabling motion control commands for controlling the medical apparatus 9. The medical apparatus 9 comprises three mobile components 3a, b, c. The apparatus position data of components 3a, b, c may be ascertained by the control unit 2 and verified by an evaluation unit 6. The control unit 2 of the safety apparatus may comprise the evaluation unit 6. Once the motion control command has been enabled by the evaluation unit 6, the control unit 5 of the medical apparatus 9 may be configured to transmit one and/or a plurality of motion control commands to components 3a, b, c of the medical apparatus 9 via a cable connection and/or a wireless connection. The control unit 2 of the safety device may be configured to send a signal to an output unit 8 in order to cause output of a warning signal. In particular, the output unit 8 may output a warning signal in the form of a sound. The output unit 8 may additionally be configured to output a signal, in particular a sound, in the event of enabling of a motion control command and/or of a change in state of the dead man's device.

The medical apparatus 9 may, for example, be an imaging apparatus, in particular a computed tomography device or a magnetic resonance apparatus. The components of the medical apparatus 9 may, for example, be ceiling mounted and swivelable monitors 3a and/or radiation protection apparatuses, patient couches 3b and/or the C-arm of a computed tomography device and/or a movable imaging component 3c.

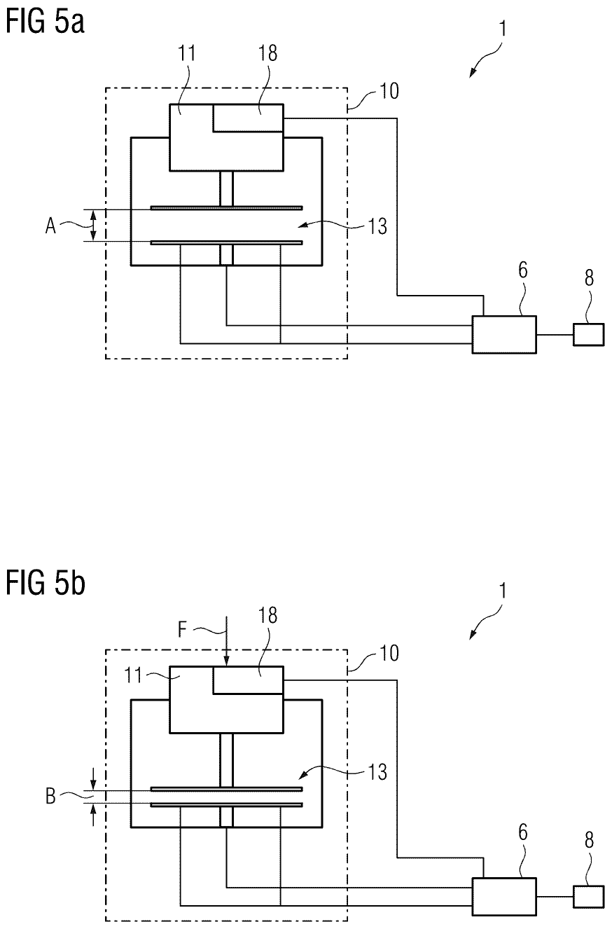

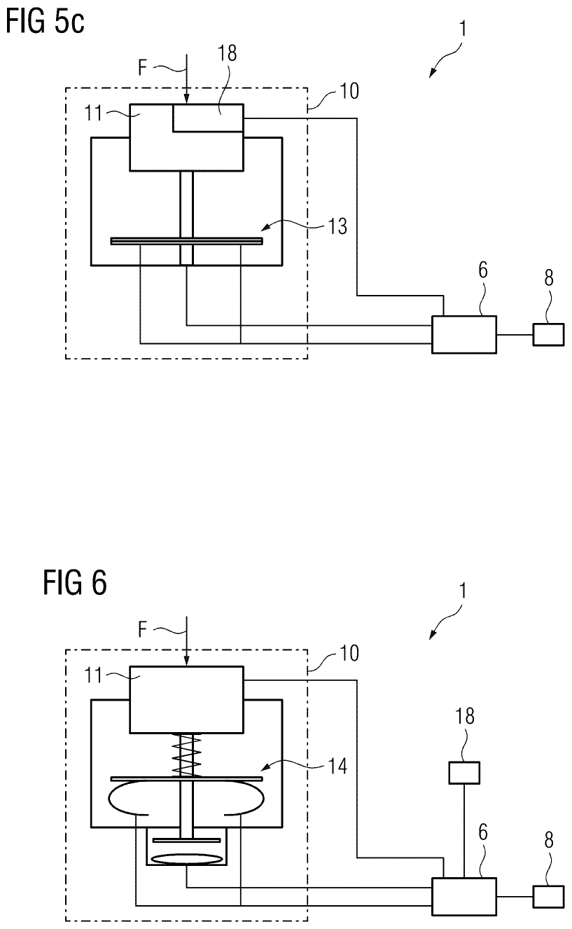

FIGS. 5a to 5c and 6 are schematic representations of the safety apparatus 1 with a dead man's device 10 in different states and embodiments. In FIGS. 5a to 5c, the dead man's device 10 comprises the safety device 1, a capacitive switch 13, and a sensor 12 as the operating sensor unit 18. The pressure sensor 12 is integrated in the operating element 11. In FIG. 6, one embodiment of a dead man's device 10 is elucidated as a mechanical and/or electronic switch 14. In addition, the embodiment comprises a further sensor 19 that is encompassed by the operating sensor unit 18. FIG. 5a shows by way of example the idle state of the dead man's device 10. FIG. 5b shows by way of example the enabling state and FIG. 5c the blocking state of the dead man's device 10.

The idle state of the dead man's device 10 may for example, as shown in FIG. 5a, be defined by a spacing A of two elements of a capacitive switch 13. The spacing of the elements, for example conductive plates, is proportional to the operating intensity, i.e., the force with which the operator actuates the operating element of the dead man's device. When a capacitive switch 13 is used, the operating intensity may also be denoted operating capacitance. The operating capacitance can be determined via the spacing A of the elements of the capacitive switch 13. In this state, the dead man's device 10 can transmit dead man's information that describes this state to the evaluation unit 6. The dead man's information may for example comprise an “off”/“0” signal and/or a value that describes the electrical capacitance.

The enabling state of the dead man's device 10 may for example, as shown in FIG. 5b, be defined by a spacing B of two elements of a capacitive switch 13. If the spacing B of the two elements of the capacitive switch 13 is reached and/or exceeded by operation, the dead man's device 10 is in the enabling state. In this state, the dead man's device 10 can transmit dead man's information that describes this state to the evaluation unit 6. The dead man's information may for example comprise an “on”/“1” signal and/or a value that describes the electrical capacitance.

The blocking state of the dead man's device 10 may for example, as shown in FIG. 5c, be defined by contact and/or spacing of two elements of a capacitive switch 13. In this state, the dead man's device 10 can transmit dead man's information that describes this state to the evaluation unit 6. The dead man's information may for example comprise an “off”/“0” signal and/or a value that describes the electrical capacitance. The blocking state may also be defined by falling below a spacing C (not shown) of the two elements of the capacitive switch 13.

The dead man's device 10 may additionally comprise an operating element 11. This may in particular be a head, grip and/or switch. The operating element 11 may comprise a sensor 12 that may be configured to acquire operating information, for example an operating intensity. The sensor 12 may be encompassed by the operating sensor unit. For example, the sensor 12 of the operating element 11 may comprise a pressure sensor that is capable of measuring an operating pressure by an operator as the operating intensity. The dead man's information may comprise the operating intensity acquired by the sensor 12. The safety apparatus may, as in FIG. 6, comprise a further sensor 19. This may, for example, be an optical sensor. The operating sensor unit 18 may comprise both a sensor 12 and a sensor 19. The sensors 12, 19 encompassed by the operating sensor unit 18 may comprise one or more extensometer strips, pressure sensors involving a change in resistance (force-sensitive resistor) and/or further sensors.

FIG. 6, like FIG. 5b, shows the enabling state of the dead man's device 10. However, in contrast with FIGS. 5a to 5c, the dead man's apparatus 10 takes the form of a mechanical switch. For example, as shown, electrical contact of two elements of a mechanical switch 14 may define the enabling state and contact of two (further) elements the blocking state. The safety apparatus also comprises an additional sensor 19 for acquiring the operating information. The sensor 19 is encompassed by the operating sensor unit 19. The sensor 19 may be directly connected to the evaluation unit 6.

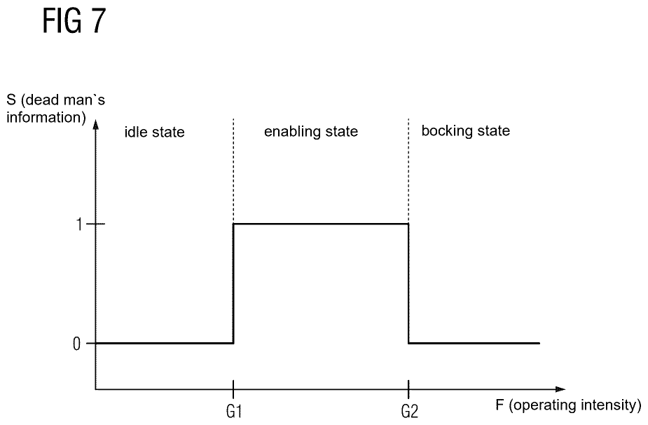

FIG. 7 is the representation of a function that elucidates the correlation between the dead man's information S and the operating intensity F of the dead man's device. In other words, FIG. 7 elucidates the relationship between the states of the dead man's device (and when said states occur due to operation of the dead man's device) and the output information from the dead man's device, in particular the dead man's information. FIG. 7 elucidates the embodiment shown in FIGS. 5a-c. Below a limit value G1 of the operating intensity F illustrated as a force, the dead man's apparatus in the idle state. From the limit value G1 up to a second limit value G2 of the operating intensity F, the dead man's device switches into the enabling state. From the limit value G3 of operating intensity F, the dead man's device is in the blocking state. The dead man's device is configured to output a 0 signal as the dead man's information in the idle state and in the blocking state. In the enabling state, the dead man's device returns a 1 signal as the dead man's information.

It should finally once again be noted that the aspects of the safety apparatus and medical apparatus described in detail above are merely exemplary embodiments which can be modified in the most varied manner by a person skilled in the art without departing from the scope of the invention. Furthermore, use of the indefinite article “a” or “an” does not rule out the possibility of a plurality of the features in question also being present. Likewise, the term “unit” does not rule out the possibility of the components in question consisting of a plurality of interacting subcomponents which may optionally also be spatially distributed.

Independent of the grammatical term usage, individuals with male, female or other gender identities are included within the term.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood “comprises,” “comprising,” “includes,” and/or that the terms “including,” when i specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the order, reverse depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as a data processing facility) or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the invention has been illustrated and described in detail by the preferred exemplary embodiments, it is not limited by the disclosed examples and a person skilled in the art can derive other variation herefrom without departing from the protective scope of the invention.

1. A safety apparatus for enabling motion control commands for controlling a medical apparatus, the safety apparatus comprising:
a controller configured to control a movement of at least a component of the medical apparatus by an operator, wherein the controller includes a dead man's device configured to output dead man's information as a function of a state of the dead man's device;
an operating sensor configured to output operating information as a function of an operation of the controller by the operator; and
an evaluator configured to verify an enablement of a motion control command based on the dead man's information and the operating information.

2. The safety apparatus of claim 1, wherein the safety apparatus device further comprises:
a motion detector configured to output motion information as a function of a movement of the operator, and wherein the evaluator is configured to verify the enablement of the motion control command based on the motion information.

3. The safety apparatus of claim 2, wherein the evaluator is configured to receive apparatus position data and to verify the enablement of the motion control command based on the apparatus position data, the apparatus position data describes the movement of at least the component of the medical apparatus.

4. The safety apparatus of claim 3, wherein the evaluator is configured to detect a presence of a hazardous situation for the operator based on the motion information and the apparatus position data and, to output an emergency stop control command when the hazardous situation is detected.

5. The safety apparatus of claim 1, wherein the dead man's device is configured to generate dead man's information as a function of at least three states of the dead man's device.

6. The safety apparatus of claim 1, wherein the controller includes,
an operating element, wherein one of the following states of the dead man's device is settable by way of an operating intensity of the operating element by the operator:
an idle state, wherein the dead man's device exhibits the idle state if the operating intensity is below a first specified limit value,
an enabling state, wherein the dead man's device exhibits the enabling state if the operating intensity is between the first limit value and a second limit value, and
a blocking state, wherein the dead man's device exhibits the blocking state if the operating intensity exceeds the second limit value, wherein the controller is configured to output an emergency stop control command in a presence of the blocking state of the dead man's device.

7. The safety apparatus of claim 6, wherein the motion control command is enabled by the evaluator only if the dead man's apparatus is in the enabling state.

8. The safety apparatus of claim 6, further comprising:
an output unit configured to output a warning signal to the operator in the presence of the blocking state.

9. The safety apparatus of claim 1, wherein the controller includes,
an operating element including the operating sensor, in particular at least one pressure sensor.

10. The safety apparatus of claim 1, wherein the dead man's device comprises a capacitive switch, and the capacitive switch is configured to acquire an operating capacitance of the dead man's device.

11. The safety apparatus of claim 2, wherein the evaluator is configured to receive the motion information, the operating information, the dead man's information and apparatus position data on the verification of the enablement of the motion control command, to verify a hazardous situation for the operator based on the motion information, the operating information and the apparatus position data, and the motion control command is enabled by the evaluator if the situation is not hazardous for the operator and the dead man's device is in an enabling state.

12. The safety apparatus of claim 1, further comprising:
an output unit configured to inform the operator about the enablement of the motion control command by at least one of a light signal or an audio signal.

13. A medical apparatus comprising:
the safety apparatus of claim 1.

14. The medical apparatus of claim 13, wherein the controller is configured to stop movement of the medical apparatus based on an output of an emergency stop control command by the safety apparatus.

15. The medical apparatus of claim 13, wherein the medical apparatus comprises a sensor configured to determine apparatus position data.

16. The safety apparatus of claim 1, wherein the motion control command is for controlling movement of the medical apparatus.

17. The safety apparatus of claim 8, wherein the warning signal is at least one of a light signal or an audio signal, to the operator in the presence of the blocking state.

18. The safety apparatus of claim 9, wherein the operating sensor includes at least one pressure sensor.
